(19) United States
(12) Patent Application Publication
(10) Pub. No.: US 2025/0289630 A1
(43) Pub. Date: Sep. 18, 2025

(54) DROPPER CAP

(71) Applicant: F.S.KOREA INDUSTRIES INC., Seoul (KR)

(72) Inventor: Jae Kwang HWANG, Seoul (KR)

(21) Appl. No.: 18/857,592

(22) PCT Filed: May 17, 2023
(86) PCT No.: PCT/KR2023/006682

§ 371 Date: Oct. 17, 2024

(30) Foreign Application Priority Data
Jul. 6, 2022 (KR) 10-2022-0082941

(51) Int. Cl. B65D 47/18

(52) U.S. Cl. CPC B65D 47/18

(57) ABSTRACT

According to an embodiment of the present invention, provided is a dropper cap which can be detached from a container part accommodating contents and which suctions or discharges the contents. The dropper cap comprises; an inner cap which can be detached from the container part; and an outer cap coupled to the inner cap so as to encompass at least a part of the inner cap, wherein the inner cap and the outer cap can be formed from a metal material.

### TECHNICAL FIELD

The present disclosure relates to a dropper cap.

### BACKGROUND ART

As a method for using liquid contents that require precise dosing in small amounts at a time, there is a method of individually packaging the required amount for each use in capsules or similar forms. However, such a method of individually packaging the required amount has a problem of inefficiency, as a relatively large amount of content remains inside the capsule even after a user used the content.

To overcome the problem of individual packaging, a method of storing the content in a container and drawing out and expelling the stored content through a dropper type discharging means has been devised. For example, a dropper cap structure where a dropper is attached to a cap of a container to allow the dropper to be attached to and detached from the container along with the cap has been devised.

Such a dropper cap adopts a double structure of an inner cap and an outer cap to protect the dropper more safely and improve heat resistance and sealing. However, when applying the double structure, chemicals such as adhesives are added to join the outer cap to the inner cap, causing stability issues. Moreover, if the outer cap and the inner cap are made of different materials, it becomes difficult to separate and recycle them, posing a problem in meeting recycling standards.

### DISCLOSURE

### Technical Problem

An objective of the present invention is to provide a dropper cap to overcome the above problems.

Objects of the present invention are not limited to the objects described above, and other objects that are not described will be clearly understood by a person skilled in the art from the description below.

### Technical Solution

According to an embodiment of the present invention, a dropper cap, which is detachably attached to a container part which accommodates contents to suck and discharge the contents, is provided. The dropper cap includes: an inner cap detachably attached to the container part; and an outer cap coupled to envelop at least a portion of the inner cap, wherein the inner cap and the outer cap are formed of a metal material.

### ADVANTAGEOUS EFFECT

According to an embodiment of the present invention, as both the inner cap and the outer cap are formed of the same metal material, the inner cap and the outer cap can be easily separated and disposed of or recycled integrally.

Furthermore, according to the present invention, the outer cap and the inner cap can be stably joined without the use of chemicals such as adhesives.

Additionally, according to the present invention, both the inner cap and the outer cap can be formed of a metal material, allowing the coupling of the inner cap and the outer cap without an undercut.

In addition, according to the present invention, during the detachment process from the container part or the assembly process of the inner cap and the outer cap, deformation of the the inner cap and the outer cap is prevented, ensuring stable fastening and joining.

### DESCRIPTION OF DRAWINGS

For a better understanding of the drawings cited in the detailed description of the present invention, a brief description of each drawing is provided.

FIG. 1 is a perspective view of a dropper cap according to an embodiment of the present invention.

FIG. 2 is an exploded perspective view of the dropper cap according to an embodiment of the present invention.

FIG. 3 is a sectional view of the dropper cap according to an embodiment of the present invention.

FIG. 4 is another sectional view of the dropper cap according to an embodiment of the present invention.

FIG. 5 is a perspective view of an outer cap according to an embodiment of the present invention.

FIG. 6 is a view for depicting an assembly process of the dropper cap according to an embodiment of the present invention.

FIG. 7 is a perspective view of a content container according to an embodiment of the present invention.

FIG. 8 is an exploded perspective view of the content container according to an embodiment of the present invention.

### BEST MODE

According to an embodiment of the present invention, a dropper cap, which is detachably attached to a container part which accommodates contents to suck and discharge the contents, is provided. The dropper cap includes: an inner cap detachably attached to the container part; and an outer cap coupled to envelop at least a portion of the inner cap, wherein the inner cap and the outer cap are formed of a metal material.

Moreover, at least one fitting protrusion is formed on the inner circumferential surface of the outer cap to closely fit the outer circumferential surface of the inner cap, and the outer cap and the inner cap are coupled together to be synchronously rotatable due to the close fitting.

Furthermore, the dropper cap further includes a dropper part penetrating through the inner cap and the outer cap and extending into the inside of the container part.

Additionally, the dropper part includes: a suction tube extending to a predetermined length in a state penetrated lengthwise; and a pressure part coupled to the top of the suction tube to induce the intake and discharge of the contents by changing volume according to pressure, wherein a first wing part and a second wing part are formed on the outer side of the pressing part to protrude and to be spaced apart from each other vertically, and at least one of the inner cap and the outer cap is inserted between the first wing part and the second wing part, so that the pressing part is fixed to at least one of the inner cap and the outer cap.

In addition, a separation space is formed between the top of the inner cap and the top of the outer cap, and the first wing part is positioned within the separation space.

Moreover, at least a portion of the bottom of the outer cap is folded inward, and the inner cap is inserted into the folded outer cap so that the inner cap is fixed to the outer cap.

Furthermore, the outer cap includes: a shielding area provided to envelop at least a portion of the outer circumferential surface of the inner cap; and a folding area provided at the bottom of the shielding area to be foldable, wherein the folding area is formed to be thinner than the shielding area.

Additionally, the inner cap comprises: a coupling area detachably coupled to the container part; and an insertion area provided at the bottom of the coupling area and inserted into the folded outer cap, and the insertion area is formed to be thinner than the coupling area.

In addition, the inner circumferential surface of the insertion area is in close contact with the folding area.

### MODE FOR INVENTION

Hereinafter, exemplary embodiments according to the present invention are described in detail with reference to the accompanying drawings. Moreover, a method of configuring and using a device according to an embodiment of the present invention will be described in detail with reference to the contents described in the accompanying drawings. The same reference numbers or symbols presented in each drawing represent parts or components performing substantially the same function. For convenience of description, the directions of up, down, left, and right are based on the drawings, and the scope of the present invention is not necessarily limited to the directions.

It will be understood that terms including ordinal numbers such as ‘first’ and ‘second’ may be used to describe various components, but the components are not limited by the terms. The terms may be used to discriminate one component from another component. For instance, the first component may be named as the second component, and on the contrary, the second component may be also named as the first component within the scope of the present invention. The terms “and/or” include combinations of multiple related items or any one of multiple related items.

It will be further understood that the words or terms used in the present invention are used to describe specific embodiments of the present invention and there is no intent to limit the present invention. The singular form of the components may be understood into the plural form unless otherwise specifically stated in the context. It should be also understood that the terms of ‘include’ or ‘have’ in the specification are used to mean that there are characteristics, numbers, steps, operations, components, parts, or combinations of the steps, operations, components and parts described in the specification and there is no intent to exclude existence or possibility of other characteristics, numbers, steps, operations, components, parts, or combinations of the steps, operations, components and parts.

Throughout the specification, when a part is referred to as being ‘connected’ to another part, this includes ‘direct connection’ and ‘indirect connection’ via an intervening part. Additionally, when a certain part ‘includes’ a certain component, other components are not excluded unless explicitly described otherwise, and other components may in fact be included.

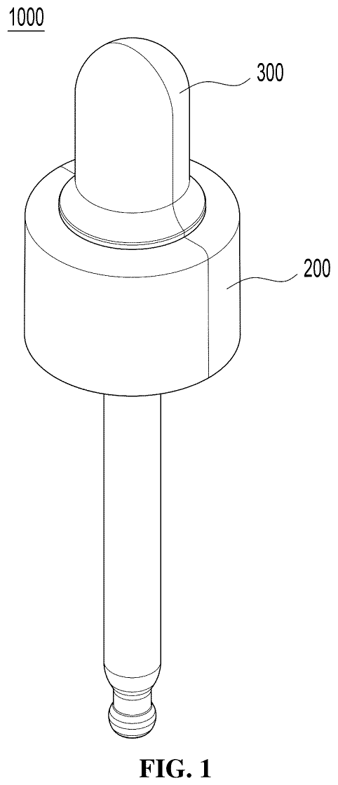

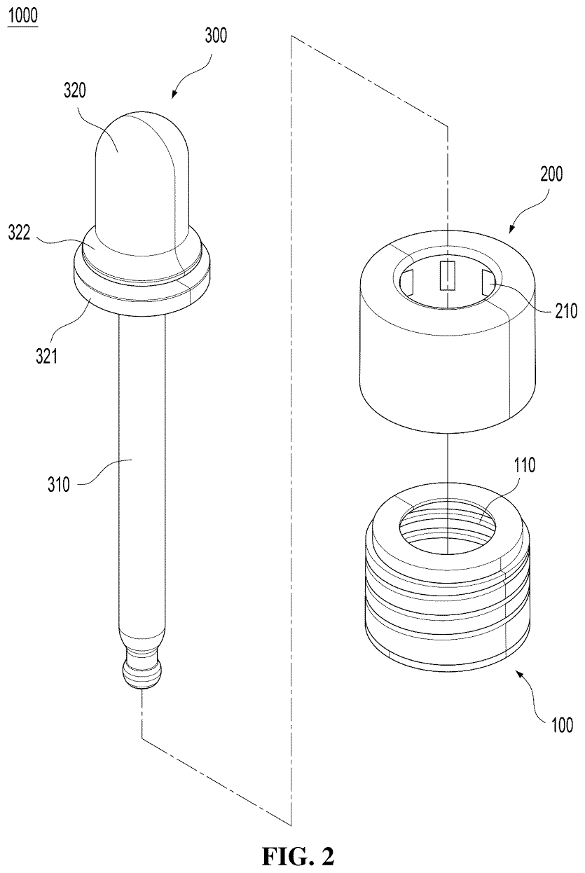

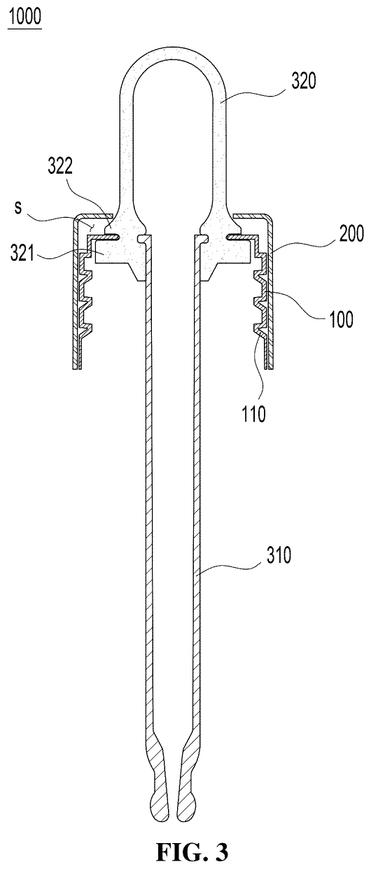

FIG. 1 is a perspective view of a dropper cap according to an embodiment of the present invention, FIG. 2 is an exploded perspective view of the dropper cap according to an embodiment of the present invention, and FIG. 3 is a sectional view of the dropper cap according to an embodiment of the present invention.

Referring to FIGS. 1 to 3, the dropper cap 1000 may include an inner cap 100, an outer cap 200, and a dropper part 300. Here, the dropper cap 1000 is detachably attached to a container part which accommodates contents and may suck in and discharge the contents.

The inner cap 100 may be detachably attached to the container part. Furthermore, the inner cap 100 can be coupled to the dropper part 300 internally and coupled to the outer cap 200 externally. The inner cap 100 supports the dropper part 300 while being attached to and detached from the container part, allowing the dropper part 300 to be indirectly coupled to and detached from the container part.

In an embodiment, the inner cap 100 may be formed of a metal material. Specifically, the inner cap 100 can be made of aluminum (Al), but is not limited thereto. For example, the inner cap 100 can be made of any one of iron (Fe), stainless steel (SUS), aluminum (Al), magnesium (Mg), and copper (Cu), or made of an alloy including at least one selected from the aforementioned materials, namely, made of one selected from various metal materials.

In the embodiment, at least one thread 110 may be formed on the inner circumferential surface of the inner cap 100. The thread 110 may be formed as at least a portion of the inner cap 100 is bent inwardly. Through the thread 110, the inner cap 100 can be attached to and detached from the container part. However, the above is merely exemplary, and besides the thread 110, various configurations capable of realizing the detachable coupling, such as a snap-fit protrusion, can be formed on the inner circumferential surface of the inner cap 100.

In the embodiment, the inner cap 100 may have a thickness of 0.48 mm or more, specifically a thickness of 0.48 to 0.52 mm, and more specifically a thickness of 0.5 mm. If the thickness of the inner cap 100 is less than 0.48 mm, the form of the thread 110 may be deformed, causing improper coupling to the container part and idle rotation.

The outer cap 200 may be detachably attached to the inner cap 100 while enveloping at least a portion of the inner cap 100. Since the outer cap 200 is coupled to envelop the inner cap 100, the structure of the inner cap 100 is not exposed to the outside, thereby forming a simple aesthetic appearance and protecting the inner cap 100 from external impacts.

In the embodiment, the outer cap 200 may be formed of a metal material. Specifically, the outer cap 200 can be made of aluminum (Al), but is not limited thereto. For instance, the outer cap 200 may be made of any one of iron (Fe), stainless steel (SUS), aluminum (Al), magnesium (Mg), and copper (Cu), or made of an alloy including at least one selected from the aforementioned materials, namely, made of one selected from various metal materials.

As described above, since the inner cap 100 and the outer cap 200 are formed of the metal material, just when the dropper part 300 is separated from the inner cap 100 and the outer cap 200, the inner cap 100 and the outer cap 200 can be integrally separated for disposal or recycling. Additionally, since both the inner cap 100 and the outer cap 200 are formed of the metal material, the inner cap 100 and the outer cap 200 can be coupled without an undercut and without using adhesives.

In the embodiment, at least one fitting protrusion 210 may be formed on the inner circumferential surface of the outer cap 200. The fitting protrusion 210 can closely fit the outer circumferential surface of the inner cap 100. By the fitting, the outer cap 200 and the inner cap 100 can be rotatably coupled. Accordingly, the outer cap 200 and the inner cap 100 can be coupled through the fitting protrusion 210 without using chemical substances such as adhesives.

In the embodiment, the outer cap 200 may have a thickness of at least 0.43 mm, specifically between 0.43 and 0.47 mm, and more specifically, a thickness of 0.45 mm. If the thickness of the outer cap 200 is less than 0.43 mm, deformation may occur when the outer cap 200 is coupled to the inner cap 100 due to insufficient rigidity, and even if coupled with the inner cap 100, the coupling may not remain stable and may come undone.

The dropper part 300 may pass through the inner cap 100 and the outer cap 200 and extend to a predetermined length. As the dropper part 300 extends to the predetermined length, the dropper part 300 can extend into the inside of the container part in the state in which the dropper cap 1000 is coupled to the container part.

In the embodiment, the dropper part 300 may include a suction tube 310 and a pressure part 320.

The suction tube 310 may extend to a predetermined length in a state penetrated lengthwise. Thus, since the suction tube 310 is penetrated lengthwise, a suction space can be formed inside the suction tube 310. The contents can be sucked into the suction space, and then discharged outside the suction space according to changes in internal pressure of the suction space.

The pressure part 320 is coupled to the top of the suction tube 310 to induce the intake and discharge of the contents by changing volume according to pressure. Specifically, when the pressure part 320 is pressed, the volume of the pressure part 320 can decrease, thereby increasing the internal pressure of the suction tube 310. Thereafter, when the pressure on the pressure part 320 is released, the volume of the pressure part 320 can increase, thereby decreasing the internal pressure of the suction tube 310. When the pressure part 320 is pressed and then released while the suction tube 310 is inserted inside the container part, the contents can be sucked into the suction space as the internal pressure of the suction tube 310 decreases. Thereafter, when the dropper cap 1000 is detached from the container part and the pressure part 320 is pressed, the contents in the suction space can be discharged to the outside depending on the increase of the internal pressure of the suction tube 310.

In the embodiment, at least one wing part 321 and 322 may be formed on the exterior of the pressure part 320. For example, on the exterior of the pressure part 320, a first wing part 321 (lower part) and a second wing part 322 (upper part) may be formed to protrude and to be spaced apart from each other vertically. In this case, at least one of the inner cap 100 and the outer cap 200 can be inserted between the first wing part 321 and the second wing part 322. Accordingly, the pressure part 320 (and the dropper part 300) can be fixed to at least one of the inner cap 100 and the outer cap 200. For example, referring to FIG. 3, an end of the inner cap 100 can be inserted and fitted between the first wing part 321 and the second wing part 322, thereby fixing the pressure part 320 to the inner cap 100. However, the present invention is not limited thereto. All ends of the inner cap 100 and the outer cap 200 may be inserted and fitted between the first wing part 321 and the second wing part 322, thereby fixing the pressure part 320 to both the inner cap 100 and the outer cap 200. So, various other embodiments can be applied.

In the embodiment, a separation space(s) may be formed between the top of the inner cap 100 and the top of the outer cap 200, and the first wing part 321 may be positioned within the separation space(s). In this case, by at least one of the inner cap 100 and the outer cap 200 inserted between the first wing part 321 and the second wing part 322, the second wing part 322 can be pushed up, thereby preventing the creation of a gap or damage to the aesthetic appearance of the dropper cap 1000.

The dropper cap 1000 illustrated in FIGS. 1 to 3 is exemplary, and various configurations can be applied according to the embodiments to which the present invention is applied.

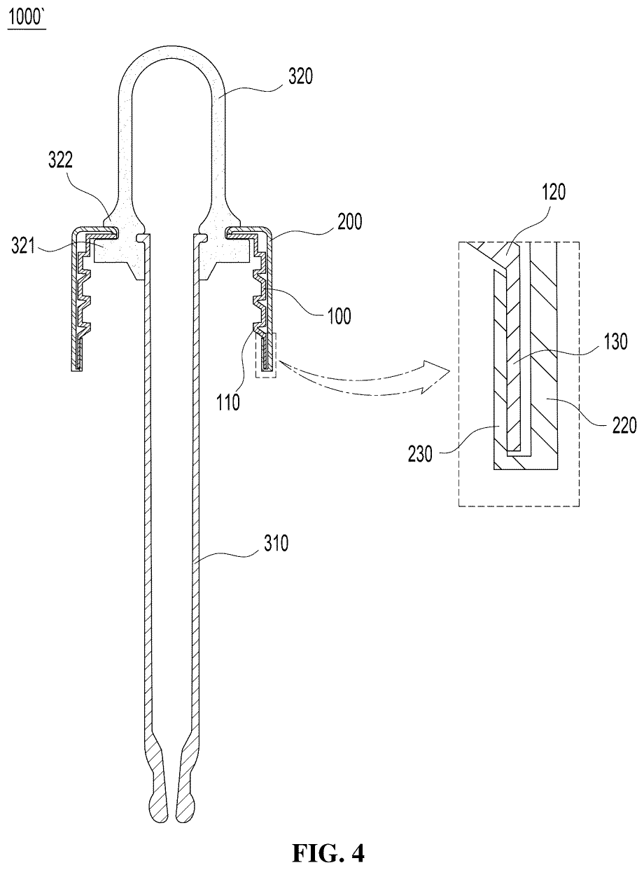

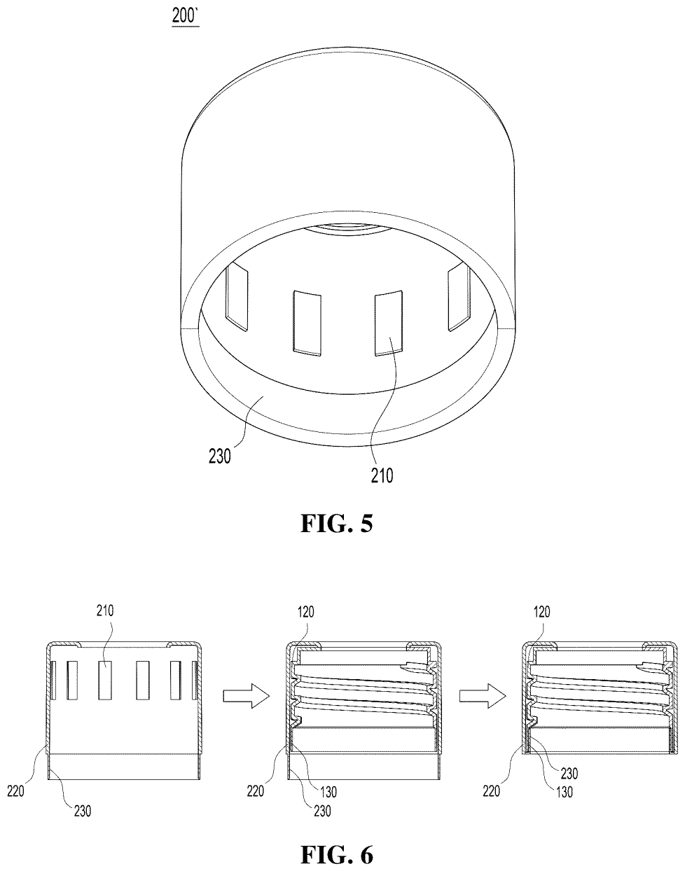

FIG. 4 is another sectional view of the dropper cap according to an embodiment of the present invention, and FIG. 5 is a perspective view of an outer cap according to an embodiment of the present invention.

Referring to FIGS. 4 and 5, a dropper cap 1000′ may include an inner cap 100′, an outer cap 200′, and a dropper part 300, and the inner cap 100′, the outer cap 200′, and the dropper part 300 can be described similarly to the dropper cap 1000 of FIGS. 1 to 3.

In this embodiment, at least a portion of the bottom of the outer cap 200′ may be folded inward, and the inner cap 100′ may be inserted into the folded outer cap 200′. By the insertion, the inner cap 100′ may be fixed to the outer cap 200′.

In this embodiment, the inner cap 100′ may include a coupling area 120 and an insertion area 130. The coupling area 120 can be detachably coupled to the container part, and the insertion area 130 is provided at the bottom of the coupling area 120 and can be inserted into the folded outer cap 200′. For example, at least a portion of the coupling area 120 may be bent inwardly to form a thread 110.

In this embodiment, the insertion area 130 may be formed to be thinner than the coupling area 120. Accordingly, even if the insertion area 130 is inserted into the folded outer cap 200′, the inner circumferential surface of the outer cap 200′ and the inner circumferential surface of the coupling area 120 can be connected smoothly.

In this embodiment, the outer cap 200′ may include a shielding area 220 and a folding area 230. The shielding area 220 may be provided to envelop at least a portion of the outer circumferential surface of the inner cap 100′, and the folding area 230 may be provided at the bottom of the shielding area 220 to be foldable.

In this embodiment, the folding area 230 may be formed to be thinner than the shielding area 220. Since the folding area 230 is formed relatively thinner, the folding area 230 can be folded more easily.

In this embodiment, the inner circumferential surface of the insertion area 130 can be in close contact with the folding area 230. Due to the close contact between the insertion area 130 and the folding area 230, the coupling force between the inner cap 100′ and the outer cap 200′ can be enhanced.

The dropper cap 1000′ illustrated in FIGS. 4 and 5 is exemplary, and various configurations may be applied according to the embodiments to which the present invention is applied.

FIG. 6 is a view for depicting an assembly process of the dropper cap according to an embodiment of the present invention.

Referring to FIG. 6, first, the inner cap 100′ can be inserted into the outer cap 200′ with the folding area 230 unfolded. After the inner cap 100′ is completely inserted, the folding area 230 can be folded so that the insertion area 130 is inserted into the outer cap 200′ to be fixed. To facilitate the folding process, the folding area 230 may be formed thinner than the shielding area 220.

The assembly process of the dropper cap 1000′ according to FIG. 6 is exemplary, and various methods may be applied according to the embodiments to which the present invention is applied.

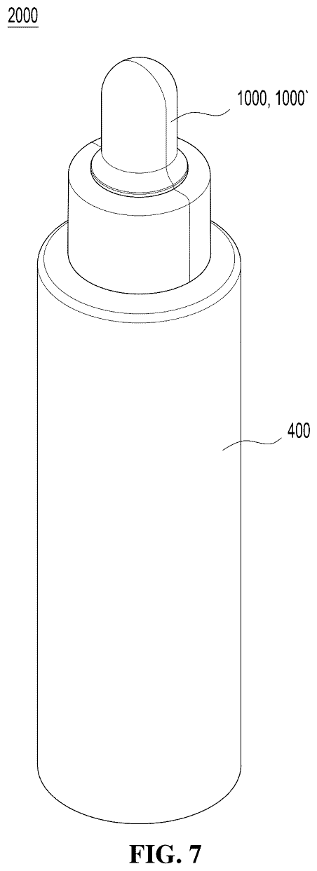

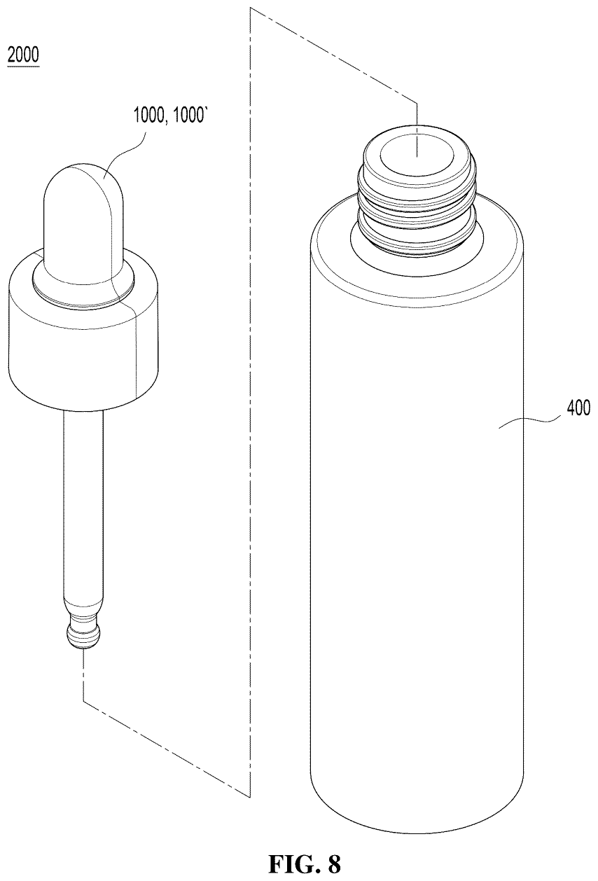

FIG. 7 is a perspective view of a content container according to an embodiment of the present invention, and FIG. 8 is an exploded perspective view of the content container according to an embodiment of the present invention.

Referring to FIGS. 7 and 8, a content container 2000 may include a dropper cap 1000 or 1000′, and a container part 400.

The dropper cap 1000 or 1000′ can be described similarly to the dropper caps 1000 and 1000′ in FIGS. 1 to 6. The dropper cap 1000 or 1000′ can be attached to and detached from the container part 400 and can suck in and discharge the content accommodated in the container part 400.

The container part 400 can accommodate content. Here, the content may be cosmetics in a liquid, gel, or powder form. For instance, the content may include lotion, milk lotion, moisture lotion, nutrition lotion, skin lotion, skin softener, skin toner, astringent, massage cream, nutrition cream, moisture cream, whitening essence, tone-up cream, sunscreen, sun cream, sun milk, BB cream, base, foundation, CC cream, concealer, blusher, shading, eye shadow, eyebrow pencil, eye cream, primer, etc. However, the present invention is not limited thereto, and other types of cosmetics, medical contents, etc., can be applied.

In this embodiment, the container part 400 may have an accommodation space formed inside to accommodate the content, and a spout formed on one side thereof to discharge the content. For instance, the container part 400 can be formed in various shapes such as a bottle, a jar, a tube, a pouch, etc.

The content container 2000 illustrated in FIGS. 7 and 8 is exemplary, and various configurations may be applied according to the embodiments to which the present invention is applied.

As disclosed above, in the drawings and specification, optimal embodiments have been disclosed. Specific terms have been used herein for the purpose of describing the present invention, but not to limit the scope of the present invention as expressed in the claims. Therefore, those skilled in the art will understand that various modifications and equivalents are possible from the present disclosure. Thus, the true scope of the technology should be determined by the technical spirit of the appended claims.

100, 100′: Inner cap 110: Thread

120: Coupling area 130: Insertion area

200, 200′: Outer cap 210: Fitting protrusion

220: Shielding area 230: Folding area

300: Dropper part 310: Suction tube

320: Pressure part 321: First wing part

322: Second wing part 400: Container part

1000, 1000′: Dropper cap 2000: Content container

1. A dropper cap, which is detachably attached to a container part which accommodates contents to suck and discharge the contents, comprising:
an inner cap detachably attached to the container part; and
an outer cap coupled to envelop at least a portion of the inner cap,
wherein the inner cap and the outer cap are formed of a metal material.

2. The dropper cap according to claim 1, wherein at least one fitting protrusion is formed on the inner circumferential surface of the outer cap to closely fit the outer circumferential surface of the inner cap, and wherein the outer cap and the inner cap are coupled together to be synchronously rotatable due to the close fitting.

3. The dropper cap according to claim 1, further comprising:
a dropper part penetrating through the inner cap and the outer cap and extending into the inside of the container part.

4. The dropper cap according to claim 3, wherein the dropper part comprises: a suction tube extending to a predetermined length in a state penetrated lengthwise; and a pressure part coupled to the top of the suction tube to induce the intake and discharge of the contents by changing volume according to pressure,
wherein a first wing part and a second wing part are formed on the outer side of the pressing part to protrude and to be spaced apart from each other vertically, and
wherein at least one of the inner cap and the outer cap is inserted between the first wing part and the second wing part, so that the pressing part is fixed to at least one of the inner cap and the outer cap.

5. The dropper cap according to claim 4, wherein a separation space is formed between the top of the inner cap and the top of the outer cap, and the first wing part is positioned within the separation space.

6. The dropper cap according to claim 1, wherein at least a portion of the bottom of the outer cap is folded inward, and
wherein the inner cap is inserted into the folded outer cap so that the inner cap is fixed to the outer cap.

7. The dropper cap according to claim 6, wherein the outer cap comprises: a shielding area provided to envelop at least a portion of the outer circumferential surface of the inner cap; and a folding area provided at the bottom of the shielding area to be foldable, and
wherein the folding area is formed to be thinner than the shielding area.

8. The dropper cap according to claim 7, wherein the inner cap comprises: a coupling area detachably coupled to the container part; and an insertion area provided at the bottom of the coupling area and inserted into the folded outer cap, and
wherein the insertion area is formed to be thinner than the coupling area.

9. The dropper cap according to claim 8, wherein the inner circumferential surface of the insertion area is in close contact with the folding area.
